(19) United States
(12) Patent Application Publication
(10) Pub. No.: US 2024/0101516 A1
(43) Pub. Date: Mar. 28, 2024

(54) SYNTHESIS OF POLYFLUORINATED ARYL AND HETEROARYL CARBOXAMIDES

(71) Applicant: BASF SE, Ludwigshafen (DE)

(72) Inventors: Rahul Kaduskar, Navi Mumbai (IN); Kailaskumar Borate, Navi Mumbai (IN); Suyog Adhav, Navi Mumbai (IN); Saravanan Thirunavukkarasu, Navi Mumbai (IN); Harish Shinde, Navi Mumbai (IN); Roland Goetz, Ludwigshafen (DE)

(21) Appl. No.: 18/272,673

(22) PCT Filed: Jan. 17, 2022
(86) PCT No.: PCT/EP2022/050851

§ 371 Date: Jul. 17, 2023

(30) Foreign Application Priority Data
Jan. 20, 2021 (EP) 21152439.2

(51) Int. Cl. C07D 213/75; C07C 231/12

(52) U.S. Cl. CPC C07D 213/75; C07C 231/12

(57) ABSTRACT

The presently claimed invention relates to a process for the preparation of mono-, di-, tri-, fluoro arylamide or hetroarylamide.

### FIELD OF THE INVENTION

The presently claimed invention relates to a process for the preparation of mono-, di- or tri-, fluoro aryl- or hetroarylamide.

### BACKGROUND OF THE INVENTION

Mono-, di- or tri-, fluoro aryl- or hetroarylamide are valuable intermediates in the agrochemical and/or pharmaceutical industry. Most often they are used as building blocks for the synthesis of fine chemicals, agrochemicals or pharmaceutical active agents.

Conventionally, amides are synthesized by coupling of moisture sensitive acid chlorides with corresponding amine partners. Aromatic amines are synthesized mainly via nitration of the aromatic compounds followed by reduction. Nitration of polyfluorinated compounds using conventional methods involves in some cases shock sensitive intermediates which creates process safety challenges. Nitration also involves use of acids as solvent/reagent and neutralization of acids used in the reaction generates large amount of aqueous waste thus process is not sustainable.

Moreover, nitration of polyfluorinated aromatic compounds and their heteroaromatic analogs is difficult to perform as it involves high energy intermediates and also generates a high effluent load, moreover regioselectivity is also a challenge.

Thus, the processes of prior art have several drawbacks, such as a high effluent load including a high metal salt load, low yields, low selectivity and critical thermal safety hazards.

Hence, there is a need to devise an improved synthetic process for the preparation of (poly)fluorinated aryl- and heteroaryl amides.

Hence, it is an object of the presently claimed invention to provide an industrially viable, sustainable and economical process for the preparation of mono-, di-, or tri-fluoro arylamides and heteroaryl amides wherein a high overall yield is achieved i.e. ≥60% or ≥80% with high selectivity. Further, the process shall also bear the advantage to be industrially viable with reduced or no safety challenges.

### SUMMARY OF THE INVENTION

Surprisingly, it has been found that mono-, di-, or tri-fluoro arylamide and heteroaryl amides are obtained in a high overall yield, i.e. ≥60% or ≥80%, with high selectivity by reacting polyfluorinated aryl halides or heteroaryl halides with an amide in the presence of a base, at least one organometallic complex wherein the organometallic complex comprises at least one mono/or polydentate phosphine ligand.

Accordingly, in one aspect, the presently claimed invention is directed to a process for the preparation of a compound of formula (I)

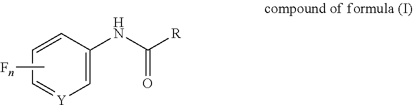

where Y is N or CH
   n is 1, 2 or 3
   R is C₁-C₁₀ alkyl, aryl or heteroaryl
comprising at least the step of
a) reacting a compound of formula (II)

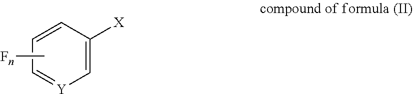

Where Y is N or CH
   X is Cl or Br
   n is 1, 2 or 3
with a compound of formula (III)

R—CO—NH₂ compound of-formula (III)
where R is C₁-C₁₀ alkyl, aryl or heteroaryl
in the presence of
i) at least one base,
ii) at least one organometallic complex
wherein the organometallic complex comprises at least one mono/or polydentate phosphine ligand

### DETAILED DESCRIPTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangea-ble under appropriate circumstances and that the embodiments of the presently claimed invention described herein are capable of operation in other sequences than described or illustrated herein.

In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of sec-onds, min, hours, days, weeks, months or even years between such steps, unless otherwise indicated in the application as set forth herein above or below.

Furthermore, the ranges defined throughout the specification include the end values as well, i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment, but may refer to the same embodiment.

Further, as used in the following, the terms “preferably”, “more preferably”, “even more preferably”, “most preferably” and “in particular” or similar terms are used in conjunction with optional features, without restricting alternative possibilities. Thus, features introduced by these terms are optional features and are not intended to restrict the scope of the claims in any way.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any one of the claimed embodiments can be used in any combination.

Further, it shall be noted that the terms “at least one”, “one or more” or similar expressions indi-cating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

The term “alkyl” refers to a linear or branched saturated hydrocarbon moiety, consisting solely of carbon and hydrogen atoms. In one embodiment from one to six carbon atoms; and in another embodiment from one to four carbon atoms; and in another embodiment one to three carbon atoms. Non-limiting examples of such substituents include methyl, ethyl, propyl (including n-propyl and isopropyl), butyl (including n-butyl, isobutyl, sec-butyl and tert-butyl), pentyl, isoamyl, hexyl and the like.

The term C₁-C₁₀ alkyl denotes a saturated linear or branched aliphatic radical with 1 to 10 carbon atoms. Examples are methyl, ethyl, n-propyl, isopropyl, n-butyl, sec-butyl, isobutyl and tert-butyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “aryl” means a carbocyclic aromatic system containing one or two rings wherein such rings may be fused. If the rings are fused, one of the rings must be fully unsaturated and the fused ring(s) may be fully saturated, partially unsaturated or fully unsaturated. The term “fused” means that a second ring is present (i.e., attached or formed) by having two adjacent atoms in common (i.e., shared) with the first ring. The term “fused” is equivalent to the term “condensed”. The aryl group may be optionally substituted as defined herein. The term “aryl” embraces aromatic radicals such as phenyl, naphthyl, tetrahydronaphthyl, indanyl, biphenyl, benzo[b][1,4]oxazin-3(4H)-onyl, 2,3-dihydro-1H indenyl and 1,2,3,4-tetrahydronaphthalenyl.

The term “heteroaryl” refers to an aromatic ring structure containing from 5 to 6 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. Examples of heteroaryl substituents include 6-membered ring substituents such as pyridyl, pyrazyl, pyrimidinyl, and pyridazinyl; and 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl.

The presently claimed invention relates to a for the preparation of a compound of formula (I)

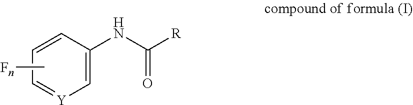

where Y is N or CH
   n is 1, 2 or 3
   R is C₁-C₁₀ alkyl, aryl or heteroaryl
comprising at least the step of
a) reacting a compound of formula (II)

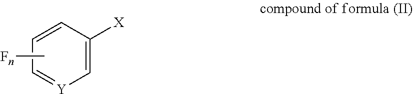

Where Y is N or CH
   X is Cl or Br
   n is 1, 2 or 3
with a compound of formula (III)

R—CO—NH₂ Compound of formula (III)
where R is C₁-C₁₀ alkyl, aryl or heteroaryl
in the presence of
   i) at least one base,
   ii) at least one organometallic complex
wherein the organometallic complex comprises at least one mono/or polydentate phosphine ligand

The process of the instant invention can be represented by the generalized reaction depicted in Scheme 1.

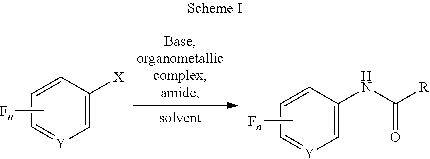

In a compound of general formula (I),

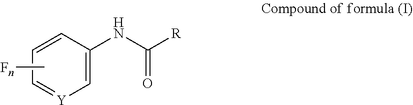

Y is N or CH,
n is 1, 2, or 3
R is C₁-C₁₀ alkyl, aryl or heteroaryl.

In a preferred embodiment, Y is N and n is 1, 2 or 3.

In a preferred embodiment when Y is N, n is 1.

In a more preferred embodiment when Y is N, n is 2 or 3.

In another preferred embodiment, Y is CH and n is 1, 2 or 3.

In a preferred embodiment when Y is CH, n is 1.

In a more preferred embodiment when Y is CH, n is 2 or 3.

In a preferred embodiment, R is selected from the group consisting of C₁-C₁₀ alkyl, aryl or heteroaryl.

In a preferred embodiment, R is selected from the group consisting of C₁-C₅ alkyl or aryl, more preferably C₁-C₂ alkyl or aryl, even more preferably methyl or phenyl.

In a compound of general formula (II)

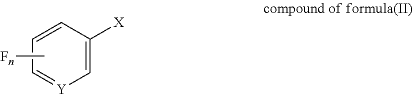

Y is N or CH, X is Cl or Br and n is 1, 2 or 3.

Preferred compounds of the general formula (II) are, the mono-, di-, tri-fluoroaryl halide is selected from the group consisting of 2 fluoro chlorobenzene, 3 Fluoro chlorobenzene, 4 fluoro chlorobenzene, 2,4 difluoro chlorobenzene, 2,2 difluoro chlorobenzene, 2,3 difluoro chlorobenzene, 3,4-difluro chlorobenzene, 3,5-difluro chlorobenzene, 1,2,3-trifluro chlorobenzene, 2 fluoro bromobenzene, 2, 4-difluoro bromobenzene, 2,3-difluoro bromobenzene, 2,3, 4-trifluoro bromobenzene.

In an embodiment of the presently claimed invention, the mono-, di-, tri-fluoro heteroaryl halide is selected from the group consisting of 2-fluoro-3-chloro pyridine, 2-Fluoro-5-chloro pyridine, 2, 3-difluoro-5-chloro pyridine, 2, 6-difluoro-5-chloro pyridine, 2,5,6-trifluoro-3-chloro pyridine, 2-fluoro-3-bromo pyridine, 2-Fluoro-5-bromo pyridine, 2, 3-difluoro-5-bromo pyridine, 2, 6-difluoro-5-bromo pyridine, 2,5,6-trifluoro-3-bromo pyridine,

In a preferred embodiment Y is N and X is Br and n is 1, 2 or 3.

Preferred compounds are 2-fluoro-3-bromo pyridine, 2-Fluoro-5-bromo pyridine, 2, 3-difluoro-5-bromo pyridine, 2, 6-difluoro-5-bromo pyridine, 2,5,6-trifluoro-3-bromo pyridine.

In another preferred embodiment Y is CH and X is Br and n is 1, 2 or 3 Preferred compounds are 2 fluoro bromobenzene, 2, 4-difluoro bromobenzene, 2,3-difluoro bromobenzene, 2,3, 4-trifluoro bromobenzene.

In a preferred embodiment, Y is CH, X is Cl and n is 1, 2 or 3.

Preferred compounds are 2 fluoro chlorobenzene, 3 fluoro chlorobenzene, 4 fluoro chlorobenzene, 2,4 difluoro chlorobenzene, 2,2 difluoro chlorobenzene, 2,3 difluoro chlorobenzene, 3,4-difluoro chlorobenzene, 3,5-difluoro chlorobenzene, 1,2,3-trifluoro chlorobenzene.

In yet another preferred embodiment, Y is N, X is Cl and n is 1, 2 or 3.

Preferred compounds are 2-fluoro-3-chloro pyridine, 2-Fluoro-5-chloro pyridine, 2, 3-difluoro-5-chloro pyridine, 2, 6-difluoro-5-chloro pyridine, 2,5,6-trifluoro-3-chloro pyridine.

In a compound of general formula (III) R is selected from the group consisting of C₁-C₁₀ alkyl, aryl or heteroaryl, preferably C₁-C₅ alkyl or aryl, more preferably C₁-C₂ alkyl or aryl.

R—CO—NH₂ compound of formula (III)

In a most preferred embodiment of the present invention, the compound of formula (III) is a compound wherein R is Methyl.

In another preferred embodiment of the present invention, the compound of formula (III) is a compound wherein R is phenyl.

In a further embodiment of the presently claimed invention, in step a) compound of formula (II) to the amide of formula (III) are employed in the range of ≥1:1 to ≤1:5.

In an embodiment of the presently claimed invention the at least one base is selected from the group consisting of alkoxides, carbonates, bicarbonates, hydroxides, amines and phosphates.

In a more preferred embodiment of the presently claimed invention, the at least one base is selected from the group consisting of sodium tert-butoxide, potassium tert-butoxide, sodium methoxide, triethylamine, trimethylamine, N-dimethylaminopyridine, 1,5-diazabicycl[4.3.0]non-ene-5 (DBN), 5-diazabicycl [5.4.0]undecene-5 (DBU), lutidine, sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, cesium fluoride, potassium hydrogen phosphate, sodium phosphate, sodium hydrogen phosphate, sodium dihydrogen phosphate, potassium dihydrogen phosphate, tributyl amine, pyridine, 1,4-diazabicyclo[2.2.2]octane (DABCO), tetramethylguanidine, sodium trifluoroacetate, potassium trifluoroacetate, cesium carbonate, cesium bicarbonate and cesium hydroxide.

In an even more preferred embodiment, the at least one base is selected from the group consisting of potassium phosphate, potassium carbonate, cesium carbonate and tetra methyl guanidine (TMG).

The organometallic complex in the form of a precursor comprises at least one mono- or polydentate phosphine ligand and at least one transition metal. The complexed transition metal is in a catalytically active valence state for the amidation reaction or can easily be transferred into a such a state during the reaction.

Preferably the complexed transition metal in the organometallic complex is palladium (Pd) and/or nickel (Ni). Most preferably the complex metal in the organometallic complex is palladium. The oxidation state of the complexed transition metal is preferably “0”.

Preferably the mono—or polydentate phosphine ligand of the organometallic complex is selected from the group consisting of 5-(di-tert-butylphosphino)-1′,3′,5′-triphenyl-1′H-1,4′-bipyrazole, dicy-clohexyl(2′,4′,6′-triisopropyl-3,6-dimethoxy-[1,1′-biphenyl]-2-yl)phosphine, bis(2-methyl-2-propa-nyl)(2′,4′,6′-triisopropyl-2-biphenylyl)phosphine, di-(1-adamantyl)-2-morpholinophenylphosphine, tributylphosphine, butyldi-1-adamantylphosphine, (5-diphenylphosphanyl-9,9-dimethylxanthen-4-yl)-diphenylphosphane, (R)-1-[(SP)-2-(diphenylphosphino)ferrocenyl]ethyldicyclohexylphosphine, dicyclohexyl-[2-[2,6-di(propan-2-yloxy)phenyl]phenyl]phosphane, bis[5-(di(1-ada-mantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropyl phos-phino)ethane, bis(2-(diphenyl-phosphino)phenyl)ether [DPE-phos], 1,2-bis(diiso-propylphosphino)ethane, 1,2-bis-(dibutylphosphino)ethane, 1,2-bis(dicyclohex-ylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(di-phenylphosphino)-butane (bppb), 2,4-bis(dicyclohexylphosphino)pentane and 1,1′-bis(diphe-nylphosphino)ferrocene (dppf), 2-Di-tert-butylphosphino-3,4,5,6-tetramethyl-2′,4′,6′-triisopropyl-1,1′-biphenyl (Me4tButylXphos) and 3,6-Dimethoxy-2′,4′,6′-tris(1-methylethyl) [1,1′-biphenyl]-2-yl]bis(1,1-dimethylethyl)phosphine (t-Bubrettphos).

Preferably the mono—or polydentate phosphine ligand of the organometallic complex is preferably biaryl phosphine ligand.

Preferably the mono—or polydentate phosphine ligand of the organometallic complex is selected from the group consisting of (R)-1-[(SP)-2-(diphenylphosphino)ferrocenyl]ethyldicyclohexylphosphine, bis(2-(diphenyl-phosphino)phenyl)ether [DPE-phos], 1,2-bis(diiso-propylphosphino)ethane, 1,2-bis-(dibutylphosphino)ethane, 1,2-bis(dicyclohex-ylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(di-phenylphosphino)-butane (bppb), 2,4-bis(dicyclohexylphosphino)pentane, 1,1′-bis(diphenylphosphino)ferrocene (dppf), 5-(di-tert-butylphosphino)-1′,3′,5′-triphenyl-1′H-1,4′-bipyrazole (Bippyphos), 3,6-Dimethoxy-2′,4′,6′-tris(1-methylethyl) [1,1′-biphenyl]-2-yl]bis(1,1-dimethylethyl)phosphine (t-Bubrettphos) dicyclohexyl(2′,4′,6′-triisopropyl-3,6-dimethoxy-[1,1′-biphenyl]-2-yl)phosphine (Brettphos), bis(2-methyl-2-propanyl)(2′,4′,6′-triisopropyl-2-biphenylyl)phosphine (tBuXPhos), dicyclohexyl-[2-[2,6-di(propan-2-yloxy)phenyl]phenyl]phosphane (Ruphos), 2-Di-tert-butylphosphino-3,4,5,6-tetramethyl-2′,4′,6′-triisopropyl-1,1′-biphenyl (Me4tButylXphos), bis[5-(di(1-adamantyl)phosphino)-1′,3′,5′-triphenyl-1′H-[1,4′]bipyrazole (AdBippyphos), bis(Ada-mantyl)(2′,4′,6′-triisopropyl-3,6-dimethoxy-2-biphenylyl)phosphine (AdBrettPhos).

More preferably the mono—or polydentate phosphine ligand of the organometallic complex is selected from the group consisting of 5-(di-tert-butylphosphino)-1′,3′,5′-triphenyl-1′H-1,4′-bipyrazole (Bippyphos), 3,6-Dimethoxy-2′,4′,6′-tris(1-methylethyl) [1,1′-biphenyl]-2-yl]bis(1,1-di-methylethyl)phosphine (t-Bubrettphos), dicyclohexyl(2′,4′,6′-triisopropyl-3,6-dimethoxy-[1,1′-biphenyl]-2-yl)phosphine (Brettphos), bis(2-methyl-2-propanyl)(2′,4′,6′-triisopropyl-2-bi-phenylyl)phosphine (tBuXPhos), dicyclohexyl-[2-[2,6-di(propan-2-yloxy)phenyl]phenyl]phosphane (Ruphos), 2-Di-tert-butylphosphino-3,4,5,6-tetramethyl-2′,4′,6′-triisopropyl-1,1′-biphenyl (Me4tButylXphos), bis[5-(di(1-adamantyl)phosphino)-1′,3′,5′-triphenyl-1′H-[1,4′]bipyrazole (AdBippyphos) bis(Adamantyl)(2′,4′,6′-triisopropyl-3,6-dimethoxy-2-biphenylyl)phosphine (AdBrettPhos).

Even more preferably, the mono- or polydentate phosphine ligand is selected from the group consisting of 2-Di-tert-butylphosphino-3,4,5,6-tetramethyl-2′,4′,6′-triisopropyl-1,1′-biphenyl (Me4tButylXphos) and 3,6-Dimethoxy-2′,4′,6′-tris(1-methylethyl) [1,1′-biphenyl]-2-yl]bis(1,1-di-methylethyl)phosphine (t-Bubrettphos).

Preferable organometallic complexes comprise the following

| Complex metal | At least on ligand selected from: |
|---|---|
| Pd(0) | 2-Di-tert-butylphosphino-3,4,5,6-tetramethyl- |
| | 2′,4′,6′-triisopropyl-1,1′-bipheny |
| | (Me4tButylXphos)) |
| Pd(0) | 3,6-Dimethoxy-2′,4′,6′-tris(1-methylethyl) |
| | [1,1′-biphenyl]-2-yl]bis(1,1-di- |
| | methylethyl)phosphine (t-Bubrettphos).) |

The organometallic complex can be prepared by reacting a suitable metal containing precursor with the respective mono- or polydentate phosphine ligand(s). Normally, the ligand is used in molar excess to the metal to be complexed. Suitable methods for the preparation of the organometallic complex are known to the person skilled in the art.

The organometallic complex can be prepared either in situ under the reaction conditions of step a) or in a separate reaction step and then be employed in step a). Pre-formation of the organometallic complex can be performed in a separate reaction vessel and the so prepared organometallic complex is then transferred into the reaction vessel of the amidation reaction, or directly in the reaction vessel of the amidation reaction.

Preferable Pd containing precursors or sources are selected from the group consisting of tetrakis(triphenylphosphine) palladium, dichlorobis(triphenylphosphine) palladium, tris(dibenzylideneacetone) dipalladium [Pd2(dba)3], bis(dibenzylideneacetone) dipalladium [Pd(dba)2], palladium acetate, dichloro(1,5-cyclooctadiene) palladium, bis[cinnamyl palladium(II)] chloride, PdCl2, and Bis(allyl)dichlorodipalladium.

In a preferred embodiment, the palladium precursor or sources are selected from the group consisting of bis(dibenzylideneacetone) dipalladium [Pd(dba)2]2, bis[cinnamyl palladium(II)] chloride, PdCl2, and Bis(allyl)dichlorodipalladium.

In a preferred embodiment, step a) is conducted in the presence of free mono- or polydentate phosphine ligand. The term “free” refers to mono- and polydentate phosphine ligands which are not complexed to a metal.

The free mono- or polydentate phosphine ligand can be the same or different from the mono- or polydentate phosphine ligand in the organometallic complex. Preferably the free mono- or polydentate phosphine ligand is the same as the mono- or polydentate phosphine ligand in the organometallic complex. However, it can be advantageously to apply a different ligand as free ligand, for example to foster ligand exchange during the Amidation reaction in step a).

Preferably, in step a) the molar ratio of the total amount transition metal to the total amount of mono- and polydentate phosphine ligands is in the range of ≥1:1 to ≤1:10, preferably ≥1:2 to ≤1:8, more preferably ≥1:2 to ≤1:6. Thus the molar ratio can be 1:2, 1:3, 1:4, 1:5, 1:6, 1:7 or 1:8, wherein 1:4 is most preferred.

More preferably, in step a) the molar ratio of the total amount of Ni and Pd to the total amount of mono- and polydentate phosphine ligands is in the range of ≥1:1 to ≤1:10, preferably ≥1:2 to ≤1:8, more preferably ≥1:2 to ≤1:6. Thus the molar ration can be 1:2, 1:3, 1:4, 1:5, 1:6, 1:7 or 1:8, wherein 1:4 is most preferred.

In case only Pd is employed in step a), the total amount of Pd to the total amount of mono- and polydentate phosphine ligands is in the range of ≥1:1 to ≤1:10, preferably ≥1:2 to ≤1:8, more preferably ≥1:2 to ≤1:6. Thus the molar ration can be 1:2, 1:3, 1:4, 1:5, 1:6, 1:7 or 1:8, wherein 1:4 is most preferred.

In a preferred embodiment of the presently claimed invention, step a) is carried out in the presence of at least one solvent which is selected from the group consisting of diethyl ether, 1,2-dimethoxy ethane, diglyme, t-butyl methyl ether, diphenyl ether, dibutyl ether, cyclopentyl methyl ether, tetrahydrofuran, 2-methyl tetrahydrofuran, diisopropylether, dioxane, benzene, xylene, toluene, hexane, pentane, cyclohexane, heptane, methylcyclohexane, esters, ethyl acetate, acetone, isopropyl acetate, n-butyl acetate, 2-butanone, n-octyl acetate, propyl acetate, tert-butylacetate, methyl isopropyl ketone, cyclohexanone, sulfolane, dimethyl carbonate, tert-butanol, tert-amyl alcohol, acetonitrile, dimethyl sulfoxide, dimethylformamide, N-methyl-2-pyrrolidone or combinations of two or more solvents.

In a more preferred embodiment of the presently claimed invention, the at least one organic solvent is selected from the group consisting of tert-butanol, tert-amyl alcohol and tetrahydrofuran.

In a preferred embodiment of the presently claimed invention, step a) is carried out a temperature in the range of ≥50° C. to ≤90° C., and more preferably a temperature in the range of ≥55° C. to ≤85° C.

In a preferred embodiment of the present invention, step a) is carried out at for a time period of ≥1 hour to ≤18 hours, preferably ≥3 hour to ≤16 hours.

In an embodiment of the presently claimed invention, the amidation of aryl mono- -di, or tri fluoro aryl chloride or mono- -di, or tri fluoro aryl bromide is carried out in the presence of acetamide, potassium phosphate, bis[cinnamyl palladium(II)] chloride, t-Bubrettphos and tert-butanol or tetrahydrofuran.

In an embodiment of the presently claimed invention, the amidation of aryl mono- -di- tri fluoro hetroaryl chloride or mono- -di, or tri fluoro heteroaryl bromide is carried out in the presence of acetamide, potassium phosphate, bis[cinnamyl palladium(II)] chloride, t-Bubrettphos and tert-butanol or tetrahydrofuran.

In an embodiment of the presently claimed invention, the amidation of aryl mono- -di- tri fluoro pyridyl chloride or mono- di, or tri fluoro pyridyl bromide is carried out in the presence of acetamide, potassium phosphate, bis[cinnamyl palladium(II)] chloride, t-Bubrettphos and tert-butanol or tetrahydrofuran.

Compound:

In an embodiment, a compound of formula (I)

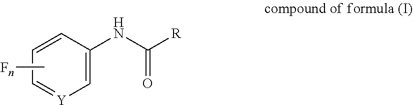

where Y is N
   n is 2 or 3
   R is C₁-C₁₀ alkyl, aryl or heteroaryl

In another embodiment of the present invention, wherein the compound of formula (I) is,

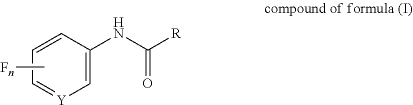

where Y is N
   n is 2 or 3
   R is CH₃.

Preferably, the compound of formula (I) is

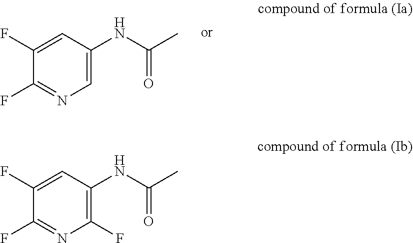

Advantages

The presently claimed invention is associated with at least one of the following advantages:
1. Substituted aryl amides are obtained in high yields with high selectivity.
2. The process of the presently claimed invention does not lead to high effluent load.
3. The process of the presently claimed invention is safe and can be easily controlled.
4. Modifying reaction conditions leads to different products, however in this case, Mono-, di-, tri-, substituted products can be obtained using the same catalyst-ligand system.
5. The process of the presently claimed invention is economical and sustainable.

In the following, there is provided a list of embodiments to further illustrate the present disclosure without intending to limit the disclosure to the specific embodiments listed below.

### Embodiments

1. A process for the preparation of a compound of formula (I)

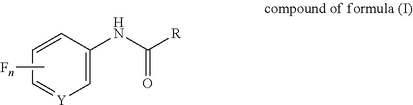

where Y is N or CH
   n is 1, 2 or 3
   R is C₁-C₁₀ alkyl, aryl or heteroaryl
comprising at least the step of
a) reacting a compound of formula (II)

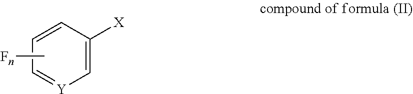

Where Y is N or CH
   X is Cl or Br
   n is 1, 2 or 3
with a compound of formula (III)

R—CO—NH₂ compound of-formula (III)
where R is C₁-C₁₀ alkyl, aryl or heteroaryl
in the presence of
   i) at least one base,
   ii) at least one organometallic complex
wherein the organometallic complex comprises at least one mono/or polydentate phosphine ligand.
2. The process according to embodiment 1, wherein the at least one organometallic complex in the form of a precursor complex comprises at least one transition metal.
3. The process according to embodiment 2, wherein the at least one transition metal is palladium or nickel.
4. The process according to embodiment 3, wherein the at least one transition metal is palladium.
5. The process according to embodiment 4 wherein, the palladium containing precursors are selected from group consisting of tetrakis(triphenylphosphine) palladium, dichlorobis(triphenylphosphine) palladium, tris(dibenzylideneacetone) dipalladium [Pd2(dba)3], bis(dibenzylideneacetone) dipalladium [Pd(dba)2], palladium acetate, dichloro(1,5-cyclooctadiene) palladium, bis[cinnamyl palladium(II)] chloride, PdCl2, and Bis(allyl)dichlorodipalladium.
6. The process according to embodiment 1 wherein, the palladium precursor or sources are selected from the group consisting of bis(dibenzylideneacetone) dipalladium [Pd(dba)2], bis[cinnamyl palladium(II)] chloride, PdCl2, and Bis(allyl)dichlorodipalladium.
7. The process according to any of the embodiments 1 to 6, wherein at least one mono/or polydentate phosphine ligand is selected from the group consisting of 5-(di-tert-bu-tylphosphino)-1′,3′,5′-triphenyl-1′H-1,4′-bipyrazole, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-bi-phenylyl)phosphine, di-(1-adamantyl)-2-morpholinophenylphosphine, tributylphosphine, butyldi-1-adamantylphosphine, (5-diphenylphosphanyl-9,9-dimethylxanthen-4-yl)-diphenylphosphane, (R)-1-[(SP)-2-(diphenylphosphino)ferrocenyl]ethyldicyclohexylphosphine, dicyclohexyl-[2-[2,6-di(propan-2-yloxy)phenyl]phenyl]phosphane, bis[5-(di(1-adaman-tyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino)ethane, bis(2-(diphenyl-phos-phino)phenyl)ether [DPE-phos], 1,2-bis(diisopropylphosphino)ethane, 1,2-bis-(dibu-tylphosphino)ethane, 1,2-bis(dicyclohexylphosphino)ethane, 1,3-bis(diisopropyl-phos-phino)propane, 1,3-bis(dicyclohexylphosphino)propane, 1,4-bis(diisopropyl-phos-phino)butane, 1,4-bis(dicyclohexylphosphino)butane, 1,4-bis(diphenylphosphino)-butane (bppb), 2,4-bis(dicyclohexylphosphino)pentane and 1,1′-bis(diphenylphosphino)ferrocene (dppf), 2-Di-tert-butylphosphino-3,4,5,6-tetramethyl-2′,4′,6′-triisopropyl-1,1′-biphenyl (Me4tButylXphos) and 3,6-Dimethoxy-2′,4′,6′-tris(1-methylethyl) [1,1′-biphenyl]-2-yl]bis(1,1-dimethylethyl)phosphine (t-Bubrettphos).
8. The process according to any of the embodiments 1 to 6, wherein at least one mono/or polydentate phosphine ligand is selected from the group consisting of of 5-(di-tert-bu-tylphosphino)-1′,3′,5′-triphenyl-1′H-1,4′-bipyrazole (Bippyphos), 3,6-Dimethoxy-2′,4′,6′-tris(1-methylethyl) [1,1′-biphenyl]-2-yl]bis(1,1-dimethylethyl)phosphine (t-Bubrettphos), dicyclohexyl(2′,4′,6′-triisopropyl-3,6-dimethoxy-[1,1′-biphenyl]-2-yl)phosphine (Brettphos), bis(2-methyl-2-propanyl)(2′,4′,6′-triisopropyl-2-biphenylyl)phosphine (tBuXPhos), dicyclohexyl-[2-[2,6-di(propan-2-yloxy)phenyl]phenyl]phosphane (Ruphos), 2-Di-tert-bu-tylphosphino-3,4,5,6-tetramethyl-2′,4′,6′-triisopropyl-1,1′-biphenyl (Me4tButylXphos), bis[5-(di(1-adamantyl)phosphino)-1′,3′,5′-triphenyl-1′H-[1,4′]bipyrazole (AdBippyphos) bis(Adamantyl)(2′,4′,6′-triisopropyl-3,6-dimethoxy-2-biphenylyl)phosphine (AdBrettPhos).
9. The process according to any of the embodiments 2 to 8, wherein the molar ratio of the total amount transition metal to the total amount of mono- and polydentate phosphine ligands is in the range of 1:1 to 1:10.
10. The process according to embodiment 9, wherein the molar ratio of the total amount transition metal to the total amount of mono- and polydentate phosphine ligands is in the range of 1:2 to 1:6.
11. The process according to embodiment 1, wherein the molar ratio of compound of formula (II) to the amide of formula (III) is in the range of ≥1:1 to ≤1:5.
12. The process according to embodiment 1, wherein R is C₁-C₅ alkyl or aryl.
13. The process according to embodiment 1, wherein R is C₁-C₂ alkyl or aryl.
14. The process according to embodiment 13, wherein R is methyl or phenyl.
15. The process according to embodiment 1, wherein X is Cl.
16. The process according to embodiment 1, wherein X is Br.
17. The process according to embodiment 1, wherein Y is N.
18. The process according to embodiment 1, wherein Y is —CH.
19. The process according to embodiment 1, wherein n is 1.
20. The process according to embodiment 1, wherein n is 2.
21. The process according to embodiment 1, wherein n is 3.
22. The process according to embodiment 1, wherein n is 2 or 3.
23. The process according to any of the embodiments 1 to 22, wherein the at least one base is selected from the group consisting of alkoxides, carbonates, bicarbonates, hydroxides, amines and phosphates.
24. The process according to embodiment 23, wherein the at least one base is selected from the group consisting of sodium tert-butoxide, potassium tert-butoxide, sodium methoxide, triethylamine, trimethylamine, N-dimethylaminopyridine, 1,5-diazabicycl[4.3.0]nonene-5 (DBN), 5-diazabicycl [5.4.0]undecene-5 (DBU), lutidine, sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, cesium fluoride, potassium hydrogen phosphate, sodium phosphate, sodium hydrogen phosphate, sodium dihydrogen phosphate, potassium dihydrogen phosphate, tributyl amine, pyridine, 1,4-diazabicyclo[2.2. 2]octane (DABCO), tetramethylguanidine, sodium trifluoroacetate, potassium trifluoroacetate, cesium carbonate, cesium bicarbonate and cesium hydroxide.
25. The process according to embodiment 24, wherein the at least one base is selected from the group consisting of potassium phosphate, potassium carbonate, cesium carbonate and tetra methyl guanidine (TMG).
26. The process according to any of the embodiments 1 to 25, wherein the process is carried out in the presence of at least one solvent which is selected from the group consisting of diethyl ether, 1, 2-dimethoxy ethane, diglyme, t-butyl methyl ether, diphenyl ether, dibutyl ether, cyclopentyl methyl ether, tetrahydrofuran, 2-methyl tetrahydrofuran, diisopropylether, dioxane, benzene, xylene, toluene, hexane, pentane, cyclohexane, heptane, methylcyclohexane, esters, ethyl acetate, acetone, isopropyl acetate, n-butyl acetate, 2-butanone, n-octyl acetate, propyl acetate, tert-butylacetate, methyl isopropyl ketone, cyclohexanone, sulfolane, dimethyl carbonate, ter-butanol, ter-amyl alcohol, acetonitrile, dimethylsulfoxide, dimethylformamide, N-methyl-2-pyrrolidone or combinations of two or more solvents.
27. The process according to embodiment 26, wherein the at least one solvent is selected from the group consisting of ter-butanol, ter-amyl alcohol and tetrahydrofuran.
28. The process according to any of the embodiments 1 to 27, wherein the process is carried out at a temperature in the range of 50° C. to 90° C.
29. The process according to any of the embodiments 1 to 27, wherein the process is carried out at a temperature in the range of 55° C. to 85° C.
30. The process according to any of the embodiments 1 to 29, wherein the process is carried out at for a time period of 1 hour to 18 hours.
31. The process according to any of the embodiments 1 to 29, wherein the process is carried out at for a time period of 3 hour to 16 hours.
32. A compound of formula (I)

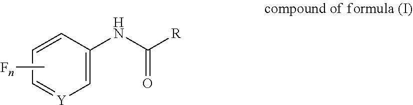

where Y is N
   n is 2 or 3
   R is C₁-C₁₀ alkyl, aryl or heteroaryl.
33. The compound according to embodiment 32, wherein the compound of formula (I) is,

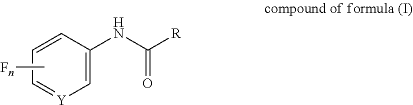

where Y is N
   n is 2 or 3
   R is CH₃.
34. The compound according to embodiment 33, wherein the compound of formula (I) is

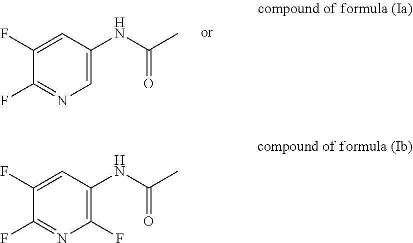

### Examples

The presently claimed invention is illustrated in detail by non-restrictive working examples which follow.

Materials

Starting Materials:
Catalyst: Bis[cinnamyl palladium(II) chloride], tris(dibenzylideneacetone) dipalladium [Pd2(dba)3] was obtained from Sigma Aldrich
Ligand: 3,6-Dimethoxy-2′,4′,6′-tris(1-methylethyl) [1,1′-biphenyl]-2-yl]bis(1,1-dimethylethyl)phosphine [t-Bubrettphos], 4,5-bis(diphenylphosphino)-9,9-dimethyl-xanthene [Xantphos]

Methods

The characterization was by coupled gas Chromatography/mass spectrometry (GC/MS),

GCMS method 1:
Instrument: Shimadzu [MS-QP2020NX], Shimadzu [GC-QP2010 Ultra]
System: GC-MS

GC Method Details:

Mobile Phase: Inert gas (Helium); Column Oven Temp: 50.0° C.; Injection Temp.: 250.00° C.; Injection Mode: Split; Flow Control Mode: Linear Velocity; Pressure: 120.0 kPa; Total Flow: 15.3 mL/min; Column Flow: 0.40 mL/min; Linear Velocity: 28.0 cm/sec; Purge Flow: 3.0 mL/min; Split Ratio: 30.0; Run Time: 8.0 min; Injection volume: 0.20-1.0 μL; Column: SH-Rxi-5 Sil MS (20 me-ter, 0.15 mm ID, 0.15 um).

Sample Preparation: In Methanol and Acetonitrile (Approx. 2 mg sample in 1.0 mL Methanol or Acetonitrile)

Oven temperature program: 50° C., hold time 1 min; rate of increase 50° C./min to 300° C. and hold time 2 min at 300° C.

MS Method Details:

Ionization source: Electron Impact Ionization; Ion Source Temp: 230.00° C.; Interface Temp.: 280.00° C.; Start m/z: 50.00; End m/z: 800.00.

Abbreviations used are: h for hour(s), min for minute(s), rt for retention time and ambient temperature for 20-25° C.

General Method for Amidation of Aryl Halide

In a three necked RBF aryl halide (6.02 mmol) was dissolved in a solvent (16 ml). Acetamide (9.03 mmol), K₃PO₄ (12.04 mmol) was added into the reaction mixture. The reaction was well purged with nitrogen gas for 5-7 min and then the catalyst Pd(cinnamyl)Cl₂ (1 mol %), ligand (4 mol %.) were added in the reaction mixture. The reaction mixture was heated in a temperature range of 60° C.-80° C. for 12 hrs and was monitored by GCMS.

Workup: The reaction mixture was cooled to room temperature. The reaction mass was filtered through celite bed and the residue was washed with 40 ml of ethyl acetate. The filtrate was evaporated to dryness and the crude compound was subjected to purification via column chromatography (elution with 10 to 70% Ethyl acetate in heptane). Column fractions was evaporated to dryness to give 0.85 g (4.49 mmol) of light brown solid compound.

General Method for Amidation of Heteroaryl Halide

In a three necked RBF heteroaryl halide (5.97 mmol) was dissolved in t-BuOH (15 ml). Acetamide (7.16 mmol), K₃PO₄ (8.95 mmol) was added into the reaction mixture. The reaction was well purged with nitrogen gas for 5-7 min and then added the catalyst Pd(cinnamyl)Cl₂ (1 mol %), ligand (4 mol %.) were added in the reaction mixture. The reaction mixture was heated to 80° C. for 3 hrs and was monitored by GCMS.

Workup: The reaction mixture was cooled to room temperature. The reaction mass was filtered through celite bed and the residue was washed with 40 ml of ethyl acetate. The filtrate was evaporated to dryness and the crude compound was subjected to purification via column chromatography (elution with 10 to 70% Ethyl acetate in heptane). Column fractions was evaporated to dryness to give 0.82 g (4.32 mmol) of light brown solid compound. Yield 72%.

Following tables provides the results of the experiments which are carried according to with example 1 or example 2 depending on the starting material employed.

TABLE 1
Conversion of Fluro Aryl chloride to fluro aryl amides

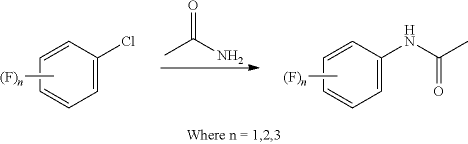

| | | | | | |
|---|---|---|---|---|---|
| | | | Temperature | Product % | |
| Ex. | Starting material | Solvent | (° C.) | conversion | Yield(%) |

Metal catalyst complex: ligand-Bis[cinnamyl palladium(II) chloride] (1 mole %); t-Bubrettphos(4
mole %)
Amide- Acetamide (1.5 equivalent)
Base: Potassium phosphate (1 equivalent)

| | | | | | |
|---|---|---|---|---|---|
| 1 | 2-Fluoro | THF | 65 | 70 | 63 |
| | chlorobenzene | | | | |
| 2 | 3-Fluoro | t-butanol | 80 | 96 | 86 |
| | chlorobenzene | | | | |
| 3 | 4-Fluoro | t-butanol | 80 | 99 | 86 |
| | chlorobenzene | | | | |
| 4 | 2,4-difluoro | t-butanol | 80 | 78 | 70 |
| | chlorobenzene | | | | |
| 5 | 2,4-difluoro | THF | 65 | 78 | 76 |
| | chlorobenzene | | | | |
| 6 | 2,3- difluoro | THF | 65 | 81 | 73 |
| | chlorobenzene | | | | |
| 7 | 3,4- difluoro | t-butanol | 80 | 95 | 91 |
| | chlorobenzene | | | | |
| 8 | 3,5- difluoro | t-butanol | 80 | 79 | 86 |
| | chlorobenzene | | | | |
| 9 | 1,2,3 trifluoro | THF | 65 | 94 | 95 |
| | chlorobenzene | | | | |

Metal catalyst complex: ligand-Pd2(dba)3 (1 mole %); Xantphos(4 mole %)
Amide- Acetamide (1.5 equivalent)
Base: Potassium phosphate (1 equivalent)

| | | | | | |
|---|---|---|---|---|---|
| 10* | 2,3,4- trifluoro- | THF | 65 | 0 | 0 |
| | chlorobenzene | | | | |

*example not as per the invention

TABLE 2
Conversion of fluoro Aryl bromide to fluoro aryl amides

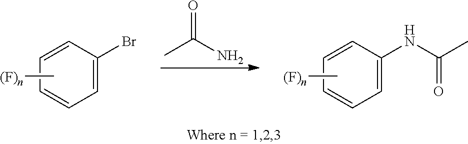

| | | | | | |
|---|---|---|---|---|---|
| | | | | Product % | |
| Ex. | Starting material | Solvent | Temperature (° C.) | conversion | Yield(%) |

Metal catalyst complex: ligand-Bis[cinnamyl palladium(II) chloride] (1 mole %); t-Bubrettphos(4
mole %)
Amide- Acetamide (1.5 equivalent)
Base: Potassium phosphate (1 equivalent)

| | | | | | |
|---|---|---|---|---|---|
| 1 | 2-Fluoro | t-butanol | 80 | 96 | 87 |
| | bromobenzene | | | | |
| 2 | 2,4 difluoro | t-butanol | 80 | 74 | 59 |
| | bromobenzene | | | | |
| 3 | 2,3 difluoro | t-butanol | 80 | 91 | 89 |
| | bromobenzene | | | | |
| 4 | 2,3,4-trifluro | t-butanol | 80 | 80 | 88 |
| | bromobenzene | | | | |

TABLE 3
Conversion of Fluro pyridyl chloride to fluoro pyridyl amide

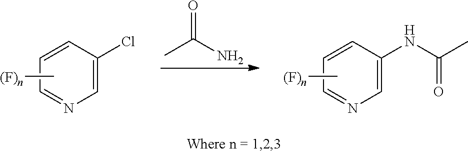

| | | | | | |
|---|---|---|---|---|---|
| | | | Temperature | Product % | |
| Ex. | Starting material | Solvent | (° C.) | conversion | Yield (%) |

Metal catalyst complex: ligand-Bis[cinnamyl palladium(II) chloride] (1 mole %); t-BubrettPhos(4
mole %)
Amide- Acetamide (1.5 equivalent)
Base: Potassium phosphate (1 equivalent)

| | | | | | |
|---|---|---|---|---|---|
| 1 | 2-Fluoro-3-Chloro | t-butanol | 80 | 75 | 63 |
| | pyridine | | | | |
| 2 | 2,3-diFluoro-5- | t-butanol | 80 | 82 | 56 |
| | Chloro pyridine | | | | |
| 3 | 2,6-diFluoro-5- | t-butanol | 80 | 99 | 78 |
| | Chloro pyridine | | | | |
| 4 | 2,5,6-triFluoro- | t-butanol | 80 | 76 | 72 |
| | Chloro pyridine | | | | |

Metal catalyst complex: ligand - Pd₂(dba)₃ (1 mole %); Xantphos(4 mole %)
Amide- Acetamide (1.5 equivalent)
Base: Potassium phosphate (1 equivalent)

| | | | | | |
|---|---|---|---|---|---|
| 5* | 2,5,6-trifluoro-Cl- | t-butanol | 80 | 0 | 0 |
| | pyridine | | | | |

*example not as per the invention

TABLE 3a
Metal catalyst complex: ligand - Bis[cinnamyl palladium(II) chloride]
(1 mole %); t-BubrettPhos(4 mole %)
Starting material: 2,5,6-trifluoro-Chloro pyridine
Solvent: t-butanol

| | | | Temperature | Product % | Yield |
|---|---|---|---|---|---|
| Ex. | Base | Amide | (° C.) | conversion | (%) |
| 1 | Potassium | Acetamide | 80 | 96 | 70 |
| | phosphate | (1.5 equivalent) | | | |
| | (1.5 equivalent) | | | | |
| 2 | Potassium | Acetamide | 80 | 80 | 65 |
| | phosphate | (1.2 equivalent) | | | |
| | (1 equivalent) | | | | |
| 3 | Potassium | Benzamide | 80 | 58 | 53 |
| | phosphate | (1.5 equivalent) | | | |
| | (1 equivalent) | | | | |

TABLE 4
Conversion of Fluro pyridyl bromide to fluoro pyridyl amide

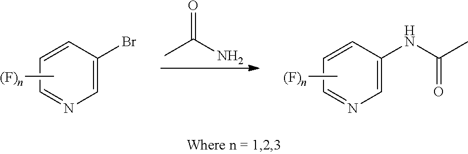

| | | | | | |
|---|---|---|---|---|---|
| | | | Temperature | Product % | |
| Ex. | Starting material | Solvent | (° C.) | conversion | Yield (%) |

Metal catalyst complex: ligand-Bis[cinnamyl palladium(II) chloride] (1 mole %); t-BubrettPhos(4
mole %)
Amide- Acetamide (1.5 equivalent)
Base: Potassium phosphate (1 equivalent)

| | | | | | |
|---|---|---|---|---|---|
| 1 | 2-Fluoro-3- | t- butanol | 80 | 84 | 85 |
| | Bromopyridine | | | | |
| 2 | 2-Fluoro-5-Bromo | t-butanol | 80 | 88 | 79 |
| | pyridine | | | | |
| 3 | 2,3-diFluoro-5- | t-butanol | 80 | 92 | 78 |
| | Bromopyridine | | | | |
| 4 | 2,6-diFluoro-5- | t-butanol | 80 | 92 | 78 |
| | Bromo pyridine | | | | |
| 5 | 2,5,6-triFluoro- | t-butanol | 80 | 95 | 72 |
| | Bromo pyridine | | | | |

1. A process for preparing a compound of formula (I) 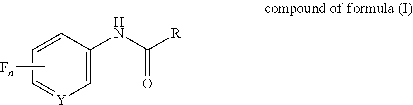
where Y is N or CH
n is 1, 2 or 3
R is C₁-C₁₀ alkyl, aryl, or heteroaryl
comprising
a) reacting a compound of formula (II) 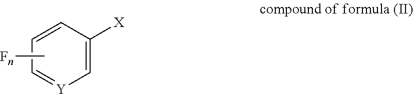
where Y is N or CH
X is Cl or Br
n is 1, 2 or 3
with a compound of formula (III)
R—CO—NH₂ compound of-formula (III)
where R is C₁-C₁₀ alkyl, aryl, or heteroaryl
in the presence of
i) at least one base,
ii) at least one organometallic complex
wherein the organometallic complex comprises at least one mono or polydentate phosphine ligand.

2. The process according to claim 1, wherein the at least one organometallic complex in the form of a precursor, comprises at least one transition metal.

3. The process according to claim 2, wherein the at least one transition metal is palladium or nickel.

4. The process according to claim 3, wherein the at least one transition metal is palladium.

5. The process according to claim 4 wherein the palladium containing precursor is selected from the group consisting of tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium [Pd(dba)2], palladium acetate, dichloro(1,5-cyclooctadiene) palladium, bis[cinnamyl palladium(II)] chloride, PdCl2, and Bis(allyl)dichlorodipalladium.

6. The process according to claim 1, wherein at least one mono or polydentate phosphine ligand is selected from the group consisting of 5-(di-tert-butylphosphino)-1′,3′,5′-triphenyl-1′H-1,4′-bipyrazole, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyldi-1-adamantylphosphine, (5-diphenylphosphanyl-9,9-dimethylxanthen-4-yl)-diphenylphosphane, (R)-1-[(SP)-2-(diphenylphosphino)ferrocenyl]ethyldicyclohexylphosphine, dicyclohexyl-[2-[2,6-di(propan-2-yloxy)phenyl]phenyl]phosphane, bis[5-(di(1-adamantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino)ethane, bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane and 1,1′-bis(diphenylphosphino)ferrocene (dppf), 2-Di-tert-butylphosphino-3,4,5,6-tetramethyl-2′,4′,6′-triisopropyl-1,1′-biphenyl (Me4tButylXphos) and 3,6-Dimethoxy-2′,4′,6′-tris(1-methylethyl) [1,1′-biphenyl]-2-yl]bis(1,1-dimethylethyl)phosphine (t-Bubrettphos).

7. The process according to claim 2, wherein a molar ratio of a total amount transition metal to a total amount of mono- and polydentate phosphine ligands is in a range of ≥1:1 to ≤1:10.

8. The process according to claim 1, wherein a molar ratio of the compound of formula (II) to the amide of formula (III) is in a range of ≥1:1 to ≤1:5.

9. The process according to claim 1, wherein R is C₁-C₅ alkyl or aryl.

10. The process according to claim 1, wherein the at least one base is selected from the group consisting of alkoxides, carbonates, bicarbonates, hydroxides, amines, and phosphates.

11. The process according to claim 10, wherein the at least one base is selected from the group consisting of sodium tert-butoxide, potassium tert-butoxide, sodium methoxide, triethylamine, trimethylamine, N-dimethylaminopyridine, 1,5-diazabicycl[4.3.0]nonene-5 (DBN), 5-diazabicycl [5.4.0]undecene-5 (DBU), lutidine, sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, cesium fluoride, potassium hydrogen phosphate, sodium phosphate, sodium hydrogen phosphate, sodium dihydrogen phosphate, potassium dihydrogen phosphate, tributyl amine, pyridine, 1,4-diazabicyclo[2.2. 2]octane (DABCO), tetramethylguanidine, sodium trifluoroacetate, potassium trifluoroacetate, cesium carbonate, cesium bicarbonate, and cesium hydroxide.

12. The process according to claim 1, wherein the process is carried out in the presence of at least one solvent selected from the group consisting of diethyl ether, 1,2-dimethoxy ethane, diglyme, t-butyl methyl ether, diphenyl ether, dibutyl ether, cyclopentyl methyl ether, tetrahydrofuran, 2-methyl tetrahydrofuran, diisopropylether, dioxane, benzene, xylene, toluene, hexane, pentane, cyclohexane, heptane, methylcyclohexane, esters, ethyl acetate, acetone, isopropyl acetate, n-butyl acetate, 2-butanone, n-octyl acetate, propyl acetate, tert-butylacetate, methyl isopropyl ketone, cyclohexanone, sulfolane, dimethyl carbonate, ter-butanol, ter-amyl alcohol, acetonitrile, dimethylsulfoxide, dimethylformamide, N-methyl-2-pyrrolidone, and combinations of two or more solvents.

13. The process according to claim 1, wherein the process is carried out at a temperature in a range of ≥50° C. to ≤90° C.

14. A compound of formula (I) 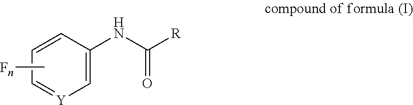
where Y is N
n is 2 or 3
R is CH₃.

15. The compound according to claim 14, wherein the compound of formula (I) is [chemical structure]
